(19) United States
(12) Patent Application Publication
(10) Pub. No.: US 2019/0391136 A1
(43) Pub. Date: Dec. 26, 2019

(54) ESTABLISHING CLINICAL-GRADE EXOSOME BANK BY DEVELOPING NOVEL ISOLATION, POTENCY PROTOCOLS, AND A NOVEL CRYOPROTECTANT SOLUTION

(71) Applicants: Milad Riazifar, Las Vegas, NV (US); Rick Saga, Las Vegas, NV (US)

(72) Inventors: Milad Riazifar, Las Vegas, NV (US); Rick Saga, Las Vegas, NV (US)

(21) Appl. No.: 16/282,274

(22) Filed: Feb. 21, 2019

Related U.S. Application Data
(60) Provisional application No. 62/633,081, filed on Feb. 21, 2018.
(60) Provisional application No. 62/643,962, filed on Mar. 16, 2018.
(60) Provisional application No. 62/643,974, filed on Mar. 16, 2018.

(51) Int. Cl. G01N 33/50; G01N 1/34; A01N 1/02

(52) U.S. Cl. CPC G01N 33/5088; A01N 1/0221; G01N 1/34

(57) ABSTRACT

A new way of establishing clinical-grade exosome banks includes breakthroughs on three fundamental pre-requisite criteria. These three breakthroughs are developing a novel protocol for isolating exosomes, developing a simple and rapid test to predict potency, and using a novel cryoprotectant solution, trehalose solution, to store exosomes. Conditioned media culture is clarified using the SYR2-U20 KR2i automated TFF system. The culture then undergoes two distinct concentration processes, after which the exosomes can be concentrated into 1 ml. Furthermore, a simple and rapid test can predict the potency of mesenchymal stem cell, derived exosomes in T cell suppression assay. A higher percentage of CD90 has been found to be correlated with higher suppression activity. These tests can be used for quality controls in clinical trial. Furthermore, the usage of trehalose solution increases the stability of exosomes and keeps their potencies over long periods of time.

### CROSS REFERENCE TO RELATED APPLICATIONS

Not applicable.

### STATEMENT REGARDING FEDERALLY SPONSORED RESEARCH OR DEVELOPMENT

Not applicable.

### FIELD OF THE INVENTION

The present invention relates to: (A) a high throughput method of isolation, (B) a thorough quality control procedure to ensure the quality of exosome frozen down, and (C) a cryoprotective solution is needed to ensure the stability of exosomes in below zero temperatures over long periods of time, and (D) methods to engineer exosomes to make them more potent and subsequently store them in the inventive bank.

### BACKGROUND OF THE INVENTION

Rigorous in vitro and in vivo testing must precede approval of exosome-based therapeutics. There are currently shortcomings in the in vitro assays used to study exosome-based products, from quality control to Mechanism of action. These assays are particularly important in clinical trial settings to ensure that prepared exosomes exert therapeutic effects before using them in humans.

CD90 is broadly used to identify MSCs since CD90 is highly expressed in all MSCs, irrespective of the source, and it is a good marker for CFU-F enrichment. High CD90 expression has also been related to the undifferentiated status of MSCs, since a decrease in CD90 level can be correlated with the temporal lineage commitment in vitro.

CD90, or Thy-1, is a 25-37 KDa glycosylphosphatidylinositol (GPI)-anchored glycoprotein. CD90 was first detected in mice T cells and later found to be expressed in thymocytes, T cells, neurons, hematopoietic stem cells, cancer stem cells, endothelial cells, and fibroblasts. Although it has been shown that CD90 is conserved among different species, its function seems to vary according to cell type. CD90 has been reported to participate in T-cell activation, neuritis outgrowth modulation, vesicular release of neurotransmitter at the synapse, astrocyte adhesion, apoptosis in carcinoma cells, tumor suppression, wound healing, fibrosis, and fibrogenesis. Furthermore, it regulates fibroblast focal adhesion, cytoskeleton organization, and cell migration. In mouse models, activation of CD90 expression can be observed in inflammation, wound healing, and tumor development. Recent studies suggest that CD90 has a role in oncogenesis, and it has also been proposed as a marker for cancer stem cells (CSCs) in various malignancies.

CD90 has also been associated with the immunosuppressive capacity. Decreased positivity for CD90 on human mesenchymal stromal cells (MSCs) has been associated with a loss of immunosuppressive activity by MSCs. CD90 molecule is also considered a predictive marker for inhibitory properties of human MSC. It might cooperate with HLA molecule in regulating suppressive properties of hMSC.

### SUMMARY OF THE INVENTION

In order to prepare any exosome bank, many essential protocols are needed including (A) a high throughput method of isolation and (B) a thorough quality control procedure to ensure the quality of exosome frozen down. Moreover, (C) a cryoprotective solution is needed to ensure the stability of exosomes in below zero temperatures over long periods of time. To this end, the invention has developed a novel protocol to isolate exosomes from liters of conditioned media. Moreover, the invention has developed a novel test, which predicts the potency of exosomes with a simple rapid test. Finally, the invention proposes a novel cryoprotectant solution, which ensures the stability of exosomes in sub-zero temperatures over long time. And, (D) methods to engineer exosomes are provided to make them more potent and subsequently store them in the inventive bank.

### BRIEF DESCRIPTION OF THE DRAWINGS

FIG. 1 is a graph showing Flow Cytometry for exosomes from three donors, with CD 63 confirmation.

FIG. 2 is a graph showing Flow Cytometry for exosomes from three donors, with CD 81 confirmation.

FIG. 3 is a graph showing Flow Cytometry for exosomes from three donors, with CD 90 confirmation. This figure includes a lower panel containing Suppression assay performed for three different donors.

### DETAILED DESCRIPTION OF THE INVENTION

In order to prepare any exosome bank, many essential protocols are needed including (A) a high throughput method of isolation and (B) a thorough quality control procedure to ensure the quality of exosome frozen down. Moreover, (C) a cryoprotective solution is needed to ensure the stability of exosomes in below zero temperatures over long periods of time. To this end, the invention has developed a novel protocol to isolate exosomes from liters of conditioned media. Moreover, the invention has developed a novel test, which predicts the potency of exosomes with a simple rapid test. Finally, the invention proposes a novel cryoprotectant solution, which ensures the stability of exosomes in sub-zero temperatures over long time. And, (D) methods to engineer exosomes are provided to make them more potent and subsequently store them in the inventive bank.

The invention is described hereunder, with regard to the following figures.

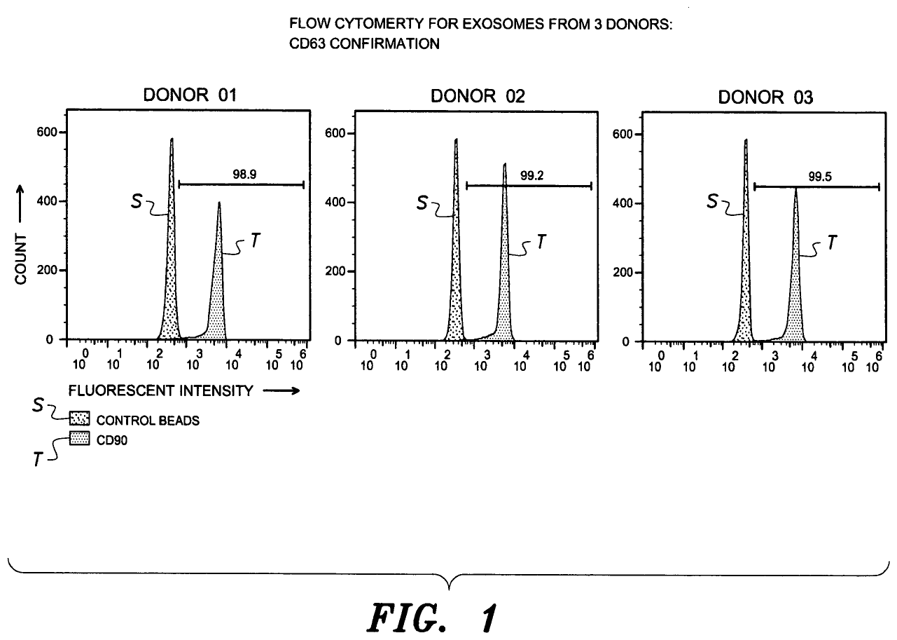

FIG. 1 is a graph showing Flow Cytometry for exosomes from three donors, with CD 63 confirmation.

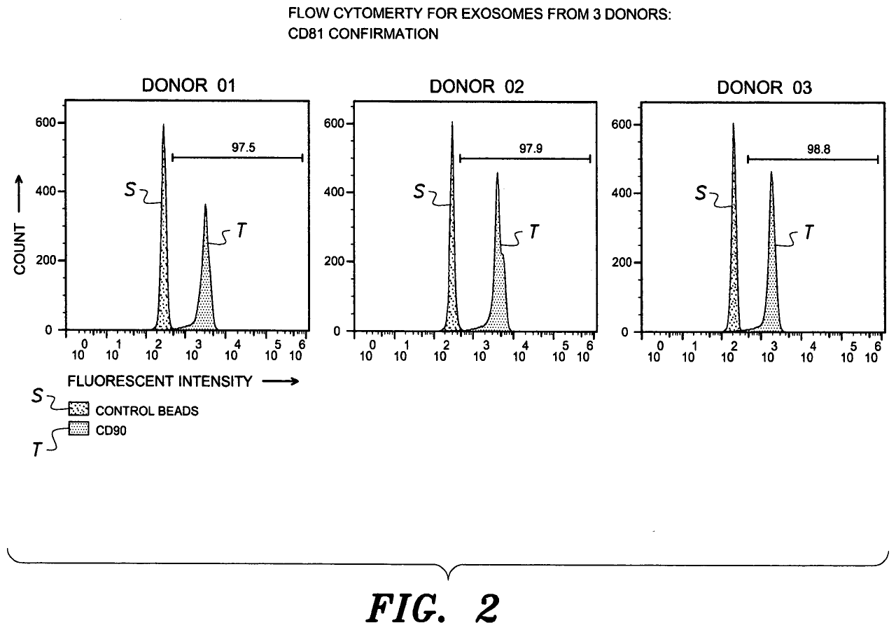

FIG. 2 is a graph showing Flow Cytometry for exosomes from three donors, with CD 81 confirmation.

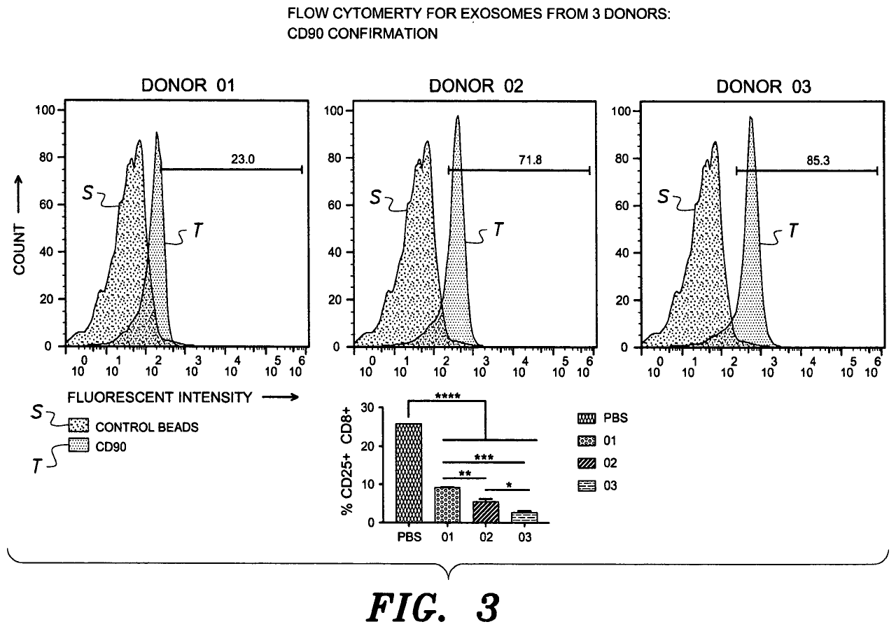

FIG. 3 is a graph showing Flow Cytometry for exosomes from three donors, with CD 90 confirmation. This figure includes a lower panel containing Suppression assay performed for three different donors.

A. Developing a Novel Protocol for Isolating Exosomes from Liters of Conditioned Media and Concentrating it into Less than 1 ml.

Exosomes are defined as small, lipid-bilayer-enclosed vesicles with a diameter of approximately 40-100 nm that are released into the extracellular milieu as a result of the fusion of intracellular multivesicular bodies (MVB) with the plasma membrane. Thus, typical exosome associated proteins (e.g. CD63, CD81 and Rab proteins) are also characteristic of endosome trafficking. Microvesicles represent a distinct class of extracellular vesicles that originate via external budding of the plasma membrane. They are more heterogeneous in size (100-1000 nm), and can potentially contain a broader array of cell surface proteins.

Exosomes and other extracellular vesicles have been reported to play important roles in physiological cell communication and tissue homeostasis events. They facilitate the transfer of nucleic acids locally between neighboring cells, but their presence in body fluids, such as blood, urine, or breast milk also enables epigenetic reprogramming of target cells at distant sites.

Isolation of exosomes, however, requires standardized and reliable methods. Currently, exosomes are frequently enriched from biofluids or conditioned media by protocols involving steps of centrifugation and ultracentrifugation, sometimes employing additional density gradient ultracentrifugation. These procedures, however, require dedicated, expensive equipment and have been shown to yield highly aggregated exosomes, accumulated protein and protein aggregates, and other non-exosome contaminants. These obstacles can confound downstream functional assays and hinder robust translation into clinical diagnostic utility. Other protocols involving antibody-coated beads are only suitable for composition analysis and not for functional studies, since modifications of the exosome surface during the isolation process are difficult to avoid.

Various chemical agents have also been proposed to facilitate exosome isolation using reduced centrifugal force, thus obviating the requirement for ultracentrifugation. However, the ability of available reagents to distinguish exosomes from microvesicles or protein aggregates remains uncertain. Microfluidic approaches based on affinity capture or novel filter materials have been reported as well.

The proposed method of the present invention enables the efficient and simplified purification of functional exosomes may open new paths towards the production of clinical-grade exosomes.

### Example

In the invention, 600 ml of conditioned media culture is clarified using a 0.2 μm vacuum filter unit. 600× Concentration and Washing is completed by a tandem TFF process using 2 custom Spectrum Labs (a Repligen Company) flow path assemblies on the KR2i automated TFF system (Spectrumlabs). Flowpath I includes a 790 cm2 500 kD hollow fiber filter. Flowpath II contains a 20 cm2 500 kD filter.

Flowpath I is set up for initial UFDF. Clarified Culture volume (600 ml) is concentrated 6×, followed by 6 diafiltration volumes of PBS wash buffer. The sample is then further concentrated to approximately 50 ml. Concentrated sample is transferred to a 50 ml conical process reservoir (Spectrumlabs). Final Concentration is performed on Flowpath II from 50 ml-1 ml.

Once the concentrate reaches 50 ml it has to go through diafiltration and more concentration using Plasma-Lyte (CB), an FDA-approved diluent for human use, has been used to dilute MSCs for infusions into patients. Once this step is performed final concentration step brings down the 50 ml into 1 ml using the same solution. Moreover, final concentration step might be proper for further use when reaches 5 ml of less. Alternatively, once the concentrate reaches 5 ml it can go through size exclusion chromatography fro further purification. Moreover, all the cell cultures should be performed in a hallow fiber bioreactor system rather than cell culture flasks.

The entire process can be performed in approximately 2.5 hours.

### Materials:

SYR2-U20 KR2i automated TFF system

Part number: 502-E500-05-S:

Family: MiniKros Sampler

Effective Length: 20 cm

Fiber: 500 kD mPES

0.5 mmPorts: ¾″ TC×¾″ TC

Part number: C02-E500-05-S:

Family: MicroKros

Effective Length: 20 cm

Fiber: 500 kD mPES

0.5 mmPorts: MLL×FLL

Part number: ACBT500-F1N

Conical Bottom Reservoir, 500 ml, 3 dip tubes, bent inlet, KR2i, Tubing ⅛″×3

Part number: ACBT-050-C1N

Conical Bottom Reservoir, 50 ml, 3 dip tubes, KR2i, Tubing 1/16″×3

Part number: ACPM-799-01N

Polysulfone Pressure Transducer, MLL×FLL, 0-75 psi, 1/pk, Non-Sterile

Part number: ACTU-E17-25N

Extended Life Silicone Tubing Size 17, 0.25″ (6.4 mm) ID, ¼″ Hose Barb

Part number: ACTU-E13-25N

Extended Life Silicone Tubing Size 13, 0.03″ (0.8 mm) ID, 1/16″ Hose Barb

### Key Points

This is a novel protocol for high scale isolation of functional exosomes from liters of conditioned media from cells.

This relates to development of a simple and rapid test, which predicts the potency of mesenchymal stem cell, derived exosomes in T cell suppression assay

B. Development of a Simple and Rapid Test, which Predicts the Potency of Mesenchymal Stem Cell, Derived Exosomes in T Cell Suppression Assay

Rigorous in-vitro and in-vivo testing must precede approval of exosome-based therapeutics. There are currently shortcomings in the in-vitro assays used to study exosome-based products, from quality control to Mechanism of action. These assays are particularly important in clinical trial settings to ensure that prepared exosomes exert therapeutic effects before using them in humans.

CD90 is broadly used to identify MSCs since CD90 is highly expressed in all MSCs, irrespective of the source, and it is a good marker for CFU-F enrichment. High CD90 expression has also been related to the undifferentiated status of MSCs, since a decrease in CD90 level can be correlated with the temporal lineage commitment in vitro.

CD90, or Thy-1, is a 25-37 KDa glycosylphosphatidylinositol (GPI)-anchored glycoprotein. CD90 was first detected in mice T cells and later found to be expressed in thymocytes, T cells, neurons, hematopoietic stem cells, cancer stem cells, endothelial cells, and fibroblasts. Although it has been shown that CD90 is conserved among different species, its function seems to vary according to cell type. CD90 has been reported to participate in T-cell activation, neuritis outgrowth modulation, vesicular release of neurotransmitter at the synapse, astrocyte adhesion, apoptosis in carcinoma cells, tumour suppression, wound healing, fibrosis, and fibrogenesis. Furthermore, it regulates fibroblast focal adhesion, cytoskeleton organization, and cell migration. In mouse models, activation of CD90 expression can be observed in inflammation, wound healing, and tumor development. Recent studies suggest that CD90 has a role in oncogenesis, and it has also been proposed as a marker for cancer stem cells (CSCs) in various malignancies.

CD90 has also been associated with the immunosuppressive capacity. Decreased positivity for CD90 on human mesenchymal stromal cells (MSCs) has been associated with a loss of immunosuppressive activity by MSCs. CD90 molecule is also considered a predictive marker for inhibitory properties of human MSC. It might cooperate with HLA molecule in regulating suppressive properties of hMSC.

### Example

In the invention, 600 ml of conditioned media culture is clarified using a 0.2 μm vacuum filter unit. 600× Concentration and Washing is completed by a tandem TFF process using 2 custom Spectrum Labs (a Repligen Company) flow path assemblies on the KR2i automated TFF system (Spectrumlabs). Flowpath I includes a 790 cm2 500 kD hollow fiber filter. Flowpath II contains a 20 cm2 500 kD filter.

Flowpath I is set up for initial UFDF. Clarified Culture volume (600 ml) is concentrated 6×, followed by 6 diafiltration volumes of PBS wash buffer. The sample is then further concentrated to approximately 50 ml. Concentrated sample is transferred to a 50 ml conical process reservoir (Spectrumlabs). Final Concentration is performed on Flowpath II from 50 ml-1 ml.

The entire process can be performed in approximately 2.5 hours.

### Materials:

SYR2-U20 KR2i automated TFF system

Part number: 502-E500-05-S:

Family: MiniKros Sampler

Effective Length: 20 cm

Fiber: 500 kD mPES

0.5 mmPorts: ¾″ TC×¾″ TC

Part number: C02-E500-05-S:

Family: MicroKros

Effective Length: 20 cm

Fiber: 500 kD mPES

0.5 mmPorts: MLL X FLL

Part number: ACBT500-F1N

Conical Bottom Reservoir, 500 ml, 3 dip tubes, bent inlet, KR2i, Tubing ⅛″×3

Part number: ACBT-050-C1N

Conical Bottom Reservoir, 50 ml, 3 dip tubes, KR2i, Tubing 1/16″×3

Part number: ACPM-799-01N

Polysulfone Pressure Transducer, MLL×FLL, 0-75 psi, 1/pk, Non-Sterile

Part number: ACTU-E17-25N

Extended Life Silicone Tubing Size 17, 0.25″ (6.4 mm) ID, ¼″ Hose Barb

Part number: ACTU-E13-25N

Extended Life Silicone Tubing Size 13, 0.03″ (0.8 mm) ID, 1/16″ Hose Barb

### Key Points

This is a novel protocol for high scale isolation of functional exosomes from liters of conditioned media from cells.

This relates to development of a simple and rapid test, which predicts the potency of mesenchymal stem cell, derived exosomes in T cell suppression assay

B. Development of a Simple and Rapid Test, which Predicts the Potency of Mesenchymal Stem Cell, Derived Exosomes in T Cell Suppression Assay

### Example

Flow cytometry of exosomes for CD90:
1. CD63 coated magnet beads vortexed for 5 seconds.
1. 20 ul of beads taken and transferred into 1.5 ml tubes. (20 ul/antibody). Here we have CD63, CD81 and CD90. Therefore, 60 ul of beads taken out.
2. 200 ul of 0.1 HSA/PBS added to beads to wash them.
3. Beads were kept on magnet for 1 min
4. The solution on top of beads is removed while kept on magnet.
5. Add 2×109 exosomes per antibody to the beads. In this case 6×109 total for three antibodies. The exosomes are from Donors: 15, 16, 17 and 32
6. The mixture of exosome kept at 4° C. overnight.
7. Tubes which incubated 4° C. overnight are placed on magnet stand for 1 min
8. Add 100 ul of 0.1 HSA/PBS to each single tube.
9. Place the tubes on magnet stand for 1 min
10. Using a 200 ul pipette remove the supernatant
11. Add 300 ul of 0.1 HSA/PBS to each tube.
12. Divide each tube into three tubes each containing 100 ul of beads coated exosomes
13. Add 2 ul of CD 81, CD63 and CD 90 to correspondent tubes (Beads with antibodies used as controls).
14. Mix well and incubate 45 min at RT
15. Add 100 ul of 0.1 HSA/PBS and place the tubes on magnet stand.
16. Remove the supernatant
17. Add 300 ul of PBS to each tube and run flow cytometry.

### Conclusion

Flow cytometry of CD90 is correlated with the degree that exosomes exert suppression on PBMCs. The higher the percentage of CD90 the higher the suppression will be.

### Key Points

This involves a simple test, which determine the potency of exosomes in PBMCs suppression assay.

The higher the percentage of CD90 is correlated with higher suppression activity.

This test can be used for quality controls in clinical trial.

This test can determine potency of exosomes with high precision in different donors.

C. Using Trehalose Solution to Store Exosomes

Trehalose is a natural, non-reducing disaccharide sugar contained in mushrooms, shrimps, insects and bacteria. Trehalose is widely used as a texturizer, stabilizer or humectant by the food and cosmetic industry and as a (cryo-) preservative for labile protein drugs, vaccines and liposomes as well as for cells and organs for transplantation. Multiple toxicity studies established the safety and tolerance of trehalose in mice and humans a after oral, gastric or parenteral administration. Interesting bioprotective actions offered by trehalose include its ability to stabilize proteins, cell membranes and liposomes, to decrease intracellular ice formation during freezing, and to prevent protein aggregation.

Extracellular vesicles suffer from aggregation and flocculation when stored in physiological saline solutions and degrade during freezing/thawing. It is currently advisable to proceed to biological assays immediately after collection, a restraining factor in large-scale or clinical trials. Proteins and RNAs are two important components of exosomes. Surprisingly, Trehalose has been shown to increase stability of RNA and proteins.

Key Points

Trehalose solution can be used to store exosomes in exosome bank.

Use of trehalose increases the stability of exosomes and keep their potencies over long periods of time.

The invention provides a new way of establishing clinical-grade exosome banks by making breakthroughs on three fundamental pre-requisite criteria. These three breakthroughs are developing a novel protocol for isolating exosomes, developing a simple and rapid test to predict potency, and using a novel cryoprotectant solution, trehalose solution, to store exosomes.

In the first breakthrough, previous procedures for isolating exosomes have required dedicated and expensive equipment, which still yield highly aggregated exosomes, accumulated protein and protein aggregates, and other non-exosome contaminants. The invention proposes a method that enables efficient and simplified purification of functional exosomes and opens new paths towards the production of clinical-grade exosomes. In the invention, conditioned media culture is clarified using the SYR2-U20 KR2i automated TFF system. The culture then undergoes two distinct concentration processes, afterwhich the exosomes can be concentrated into 1 ml.

In the second breakthrough, rigorous in-vitro and in-vivo testing must precede approval of exosome-based therapeutics. There are currently shortcomings in the in-vitro assays used to study exosome-based products, from quality control to Mechanism of action. The invention has developed a simple and rapid test for predicting the potency of mesenchymal stem cell, derived exosomes in T cell suppression assay. A higher percentage of CD90 has been found to be correlated with higher suppression activity. These tests can be used for quality controls in clinical trial.

In the third breakthrough, Trehalose is a natural, non-reducing disaccharide sugar contained in mushrooms, shrimps, insects and bacteria. Trehalose is widely used as a texturizer, stabilizer or humectant by the food and cosmetic industry and as a (cryo-) preservative for labile protein drugs, vaccines and liposomes as well as for cells and organs for transplantation. Taken that extracellular vesicles suffer from aggregation and flocculation when stored in physiological saline solutions and degrade during freezing/thawing, the invention uses trehalose solution to store exosomes in exosome banks. The usage of trehalose solution increases the stability of exosomes and keeps their potencies over long periods of time.

D) Engineering Exosomes:

The three main types of EV cargo are proteins, RNAs, and small-molecule drugs, which can be loaded into EVs by active approaches (i.e., incorporation during EV biogenesis, such as by genetic modification of the cells) and passive approaches [i.e., incorporation after EV secretion, such as by electroporation or chemical conjugation. Engineering of stem cell EVs typically takes advantage of their natural production processes and properties and further combines them with genetic or nongenetic designs to add functionalities (e.g., targeting, therapy, sensing, imaging). Receptor-ligand pairs can be used to deliver modified EVs bearing receptors that bind to the ligands on target cells, therefore reducing off-target effects while increasing efficacy.

Here, we are introducing intelligent exosomes which the inventor will call Exolntel™, SmartExo™, and/or BrilliantExo™. The trademark Exolntel™ will be defined by the inventor as covering engineered exosomes, which have targeting ability to a specific tissue and can deliver specific RNA or protein cargo to the target cells. Here, in this inventive exosome bank, the inventors are able to engineer exosomes for specific diseases and make them more potent followed by storing them in the inventive bank.

The invention being thus described, it will be evident that the same may be varied in many ways by a routineer in the applicable arts. Such variations are not to be regarded as a departure from the spirit and scope of the invention and all such modifications are intended to be included within the scope of the claims.

1. A protocol for isolating exosomes from liters of conditioned media and concentrating it into less than 1 ml, including the steps of:
(A) performing high scale isolation of functional exosomes from liters of conditioned media from cells, and
(B) providing a simple and rapid test, which predicts the potency of mesenchymal stem cell, derived exosomes in a T cell suppression assay.

2. A simple and rapid test, which predicts the potency of mesenchymal stem cell, derived exosomes in T cell suppression assay, including the steps of:
(A) determining potency of exosomes in PBMCs suppression assay;
(B) determining a percentage of CD90, wherein a higher the percentage of CD90 is correlated with a higher suppression activity;
(C) using the results of step (B) for quality controls in clinical trial; and
(D) using the results of steps (A), (B), and (C) for determining potency of exosomes with high precision in different donors.

3. The test of claim 2, further comprising step (E) of using trehalose solution to store exosomes.

4. The test of claim 3, further comprising step (F) of providing an exosome bank, and using said Trehalose solution for storing exosomes in said exosome bank.

5. The protocol of claim 1, comprising the further step of using Trehalose solution for increasing the stability of exosomes so as to keep their potencies over long periods of time.

6. A method for providing an exosome bank containing exosomes which have been engineered to make them more potent, comprising the steps of:
(A) providing smart exosomes having the capacity to target specific tissues and cells and also deliver specific cargo; and
(B) providing an exosome bank having a capacity for storing all clinical-grade, quality controlled exosomes for therapeutic purposes.
